(19) United States
(12) Patent Application Publication
(10) Pub. No.: US 2012/0095313 A1
(43) Pub. Date: Apr. 19, 2012

(54) MEDICAL DEVICES THAT SUPPORT ENHANCED SYSTEM EXTENSIBILITY FOR DIABETES CARE

(75) Inventors: Robert E. Reinke, Indianapolis, IN (US); Julia Dymshitz, Carmel, IN (US); Igor Gejdos, Indianapolis, IN (US)

(73) Assignee: ROCHE DIAGNOSTICS OPERATIONS, INC., Indianapolis, IN (US)

(21) Appl. No.: 12/905,471

(22) Filed: Oct. 15, 2010

(51) Int. Cl. A61B 5/00; G06Q 10/00; G06Q 50/00

(52) U.S. Cl. 600/365; 705/2

(57) ABSTRACT

A medical device or medical software is provided that supports system extensibility for diabetes care. The medical device or software is comprised of an application and particular data structures that support diabetes care. The data structures include: a patient class that has attributes and methods associated with a person receiving medical treatment for diabetes; a patient log class that has a composition relationship with the patient class and attributes and methods that log actions taken by the patient; a treatment plan class that has a composition relationship with the patient class and attributes and methods that define a series of planned actions related to medical treatment of the patient; and an adherence class that has a composition relationship with the patient log class and attributes and methods define relationships between actions planned for the patient and actions taken by the patient. The application instantiates an object from at least one of the patient log class, the adherence class and the treatment plan class, having only external-to-the-composition knowledge of which objects are instantiated, and performs a function using the instantiated object.

### FIELD

The present disclosure relates to medical devices used for diabetes care and, more particularly, to extensibility for a system of medical devices used for diabetic care.

### BACKGROUND

Diabetes mellitus, often referred to as diabetes, is a chronic condition in which a person has elevated blood glucose levels that result from defects in the body's ability to produce and/or use insulin. There are three main types of diabetes. Type 1 diabetes usually strikes children and young adults, and may be autoimmune, genetic, and/or environmental. Type 2 diabetes accounts for 90-95% of diabetes cases and is linked to obesity and physical inactivity. Gestational diabetes is a form of glucose intolerance diagnosed during pregnancy and usually resolves spontaneously after delivery.

In 2009, according to the World Health Organization, at least 220 million people worldwide suffer from diabetes. In 2005, an estimated 1.1 million people died from diabetes. Its incidence is increasing rapidly, and it is estimated that between 2005 and 2030, the number of deaths from diabetes will double. In the United States, nearly 24 million Americans have diabetes with an estimated 25 percent of seniors age 60 and older being affected. The Centers for Disease Control and Prevention forecast that 1 in 3 Americans born after 2000 will develop diabetes during their lifetime. The National Diabetes Information Clearinghouse estimates that diabetes costs $132 billion in the United States alone every year. Without treatment, diabetes can lead to severe complications such as heart disease, stroke, blindness, kidney failure, amputations, and death related to pneumonia and flu.

Diabetes is managed primarily by controlling the level of glucose in the bloodstream. This level is dynamic and complex, and is affected by multiple factors including the amount and type of food consumed, and the amount of insulin (which mediates transport of glucose across cell membranes) in the blood. Blood glucose levels are also sensitive to exercise, sleep, stress, smoking, travel, illness, menses, and other psychological and lifestyle factors unique to individual patients. The dynamic nature of blood glucose and insulin, and all other factors affecting blood glucose, often require a person with diabetes to forecast blood glucose levels. Therefore, therapy in the form of insulin or oral medications, or both, can be timed to maintain blood glucose levels in an appropriate range.

Management of diabetes is time-consuming for patients because of the need to consistently obtain reliable diagnostic information, follow prescribed therapy, and manage lifestyle on a daily basis. Diagnostic information, such blood glucose, is typically obtained from a capillary blood sample with a lancing device and is then measured with a handheld blood glucose meter. Interstitial glucose levels may be obtained from a continuous glucose sensor worn on the body. Prescribed therapies may include insulin, oral medications, or both. Insulin can be delivered with a syringe, an ambulatory infusion pump, or a combination of both. With insulin therapy, determining the amount of insulin to be injected can require forecasting meal composition of fat, carbohydrates and proteins along with effects of exercise or other physiologic states. The management of lifestyle factors such as body weight, diet, and exercise can significantly influence the type and effectiveness of a therapy.

Management of diabetes involves large amounts of diagnostic data and prescriptive data acquired in a variety of ways: from medical devices, from personal healthcare devices, from patient-recorded logs, from laboratory tests, and from healthcare professional recommendations. Medical devices include patient-owned bG meters, continuous glucose monitors, ambulatory insulin infusion pumps, diabetes analysis software, and diabetes device configuration software. Each of these systems generates and/or manages large amounts of diagnostic and prescriptive data. Personal healthcare devices include weight scales, blood pressure cuffs, exercise machines, thermometers, and weight management software. Patient recorded logs include information relating to meals, exercise and lifestyle. Lab test results include HbA1C, cholesterol, triglycerides, and glucose tolerance. Healthcare professional recommendations include prescriptions, diets, test plans, and other information relating to the patient's treatment.

Patients with diabetes and their healthcare professionals interact with a variety of medical devices and systems to help manage the disease. For each of these differing types of medical devices, there is a need to aggregate, manipulate, manage, present, and communicate diagnostic data and prescriptive data from multiple data sources in an efficient manner to improve the care and health of a person with diabetes, so the person with diabetes can lead a full life and reduce the risk of complications from diabetes. There is also a need to aggregate, manipulate, manage, present, and communicate such diagnostic data and prescriptive data amongst the different types of medical devices.

When designing an overall system for diabetes management or an application residing on a given medical device in the system, there is a further need to identify and implement extension points in the system to support future growth. The background description provided herein is for the purpose of generally presenting the context of the disclosure.

### SUMMARY

A handheld medical device is provided that supports system extensibility for diabetes care. The medical device is comprised of an application and a particular data structure that supports diabetes care. The data structure includes: a patient class that has attributes and methods associated with a person receiving medical treatment for diabetes; a patient log class that has a composition relationship with the patient class and attributes and methods that log actions taken by the patient; a treatment plan class that has a composition relationship with the patient class and attributes and methods that define a series of planned actions related to medical treatment of the patient; and an adherence class that has a composition relationship with the patient log class and attributes and methods define relationships between actions planned for the patient and actions taken by the patient. The application instantiates an object from at least one of the patient log class, the adherence class and the treatment plan class and performs a function using the instantiated object.

Further areas of applicability of the present disclosure will become apparent from the detailed description provided hereinafter. It should be understood that the detailed description and specific examples are intended for purposes of illustration only and are not intended to limit the scope of the disclosure.

### BRIEF DESCRIPTION OF THE DRAWINGS

FIG. 1 is a diagram showing a patient and a treating clinician;

FIG. 2 is a diagram showing the patient with a continuous glucose monitor (CGM), an ambulatory durable insulin infusion pump, an ambulatory non-durable insulin infusion pump, and a diabetes manger;

FIG. 3 is a block diagram showing an exemplary diabetes management system used by patients and clinicians to manage diabetes;

FIG. 4 is a functional block diagram of a diabetes manager;

FIG. 5 is a diagram of a domain model for the diabetes care information management domain;

FIG. 6 is a class diagram for a portion of the diabetes care domain model that relates to a patient's log;

FIG. 7 is a class diagram for a portion of the diabetes care domain model that relates to a treatment plans;

FIG. 8 is a class diagram for a portion of the diabetes care domain model that relates to adherence;

FIG. 9 is a class diagram for a portion of the diabetes care domain model that relates to medical devices;

FIG. 10 is a diagram that illustrates a container class referencing an interface; and

FIG. 11 is diagram that illustrates an application that instantiates an object of the container class by referencing the interface.

The drawings described herein are for illustrative purposes only of selected embodiments and not all possible implementations, and are not intended to limit the scope of the present disclosure. Corresponding reference numerals indicate corresponding parts throughout the several views of the drawings.

### DETAILED DESCRIPTION

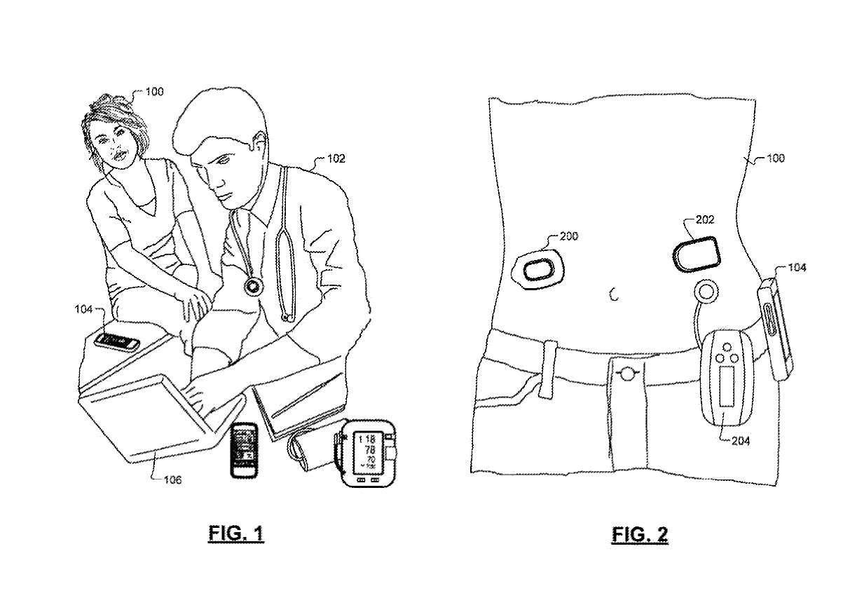

Referring now to FIG. 1, a person 100 with diabetes and a healthcare professional 102 are shown in a clinical environment. Persons with diabetes include persons with metabolic syndrome, pre-diabetes, type 1 diabetics, type 2 diabetics, and gestational diabetics and are collectively referred to as a patient. Healthcare providers for diabetes are diverse and include nurses, nurse practitioners, physicians, and endocrinologists and are collectively referred to as a clinician.

During a healthcare consultation, the patient 100 typically shares with the clinician 102 a variety of patient data including blood glucose measurements, continuous glucose monitor data, amounts of insulin infused, amounts of food and beverages consumed, exercise schedules, and other lifestyle information. The clinician 102 may obtain additional patient data that includes measurements of HbA1C, cholesterol levels, triglycerides, blood pressure, and weight of the patient 100. The patient data can be recorded manually or electronically on a handheld diabetes management device 104, a diabetes analysis software executed on a personal computer (PC) 106, and/or a web-based diabetes analysis site (not shown). The clinician 102 can analyze the patient data manually or electronically using the diabetes analysis software and/or the web-based diabetes analysis site. After analyzing the patient data and reviewing adherence of the patient 100 to previously prescribed therapy, the clinician 102 can decide whether to modify the therapy for the patient 100.

Referring now to FIG. 2, the patient 100 can use a continuous glucose monitor (CGM) 200, an ambulatory non-durable insulin infusion pump 202 or an ambulatory durable insulin infusion pump 204 (hereinafter insulin pump 202 or 204), and the handheld diabetes management device 104 (hereinafter the diabetes manager 104). The CGM 200 uses a subcutaneous sensor to sense and monitor the amount of glucose in the interstitial fluid of the patient 100 and communicates corresponding readings to the diabetes manager 104.

The diabetes manager 104 performs various tasks including measuring and recording blood glucose levels, determining an amount of insulin to be administered to the patient 100 via the insulin pump 202 or 204, receiving patient data via a user interface, archiving the patient data, etc. The diabetes manager 104 periodically receives readings from the CGM 200 indicating glucose level in the interstitial fluid of the patient 100. The diabetes manager 104 transmits instructions to the insulin pump 202 or 204, which delivers insulin to the patient 100. Insulin can be delivered in a scheduled manner in the form of basal (background) doses, which consists of multiple small doses of insulin to the patient 100 over an extended period (e.g., a day). Additionally, insulin can be delivered in the form of a bolus dose, which delivers a single larger quantity of insulin to the patient 100 to adjust for a particular situation (e.g., eating a meal).

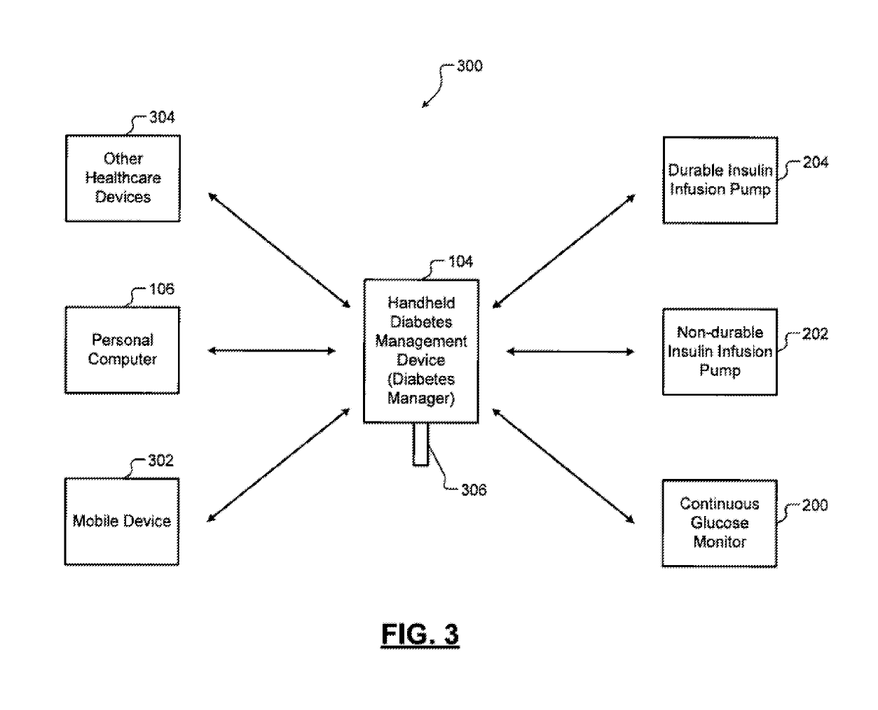

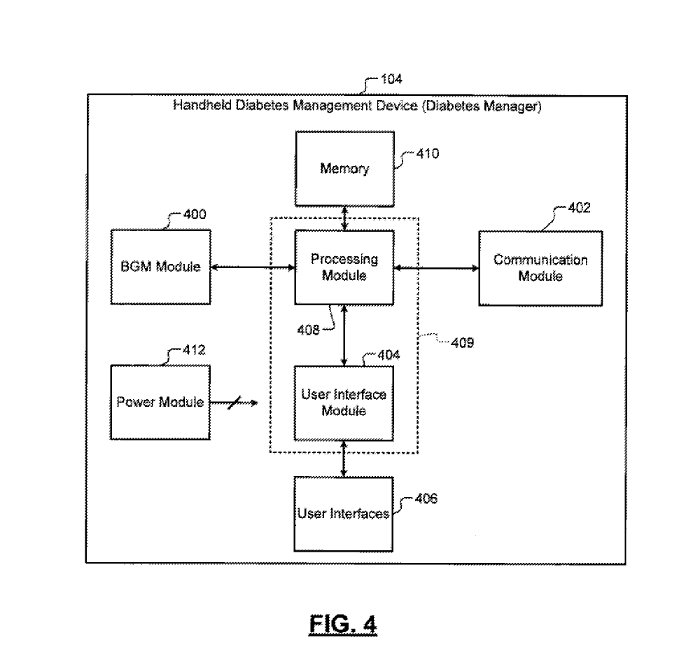

Referring now to FIG. 3, a diabetes management system 300 used by the patient 100 and the clinician 102 includes one or more of the following devices: the diabetes manager 104, the continuous glucose monitor (CGM) 200, the insulin pump 202 or 204, a mobile device 302, the PC 106 with the diabetes analysis software, and other healthcare devices 304. The diabetes manager 104 is configured as a system hub and communicates with the devices of the diabetes management system 300. Alternatively, the insulin pump 204 or the mobile device 302 can serve as the system hub. Communication between the devices in the diabetes management system 300 can be performed using wireless interfaces (e.g., Bluetooth) and/or wireline interfaces (e.g., USB). Communication protocols used by these devices can include protocols compliant with the IEEE 11073 standard as extended using guidelines provided by Continua® Health Alliance Design Guidelines. Further, healthcare records systems such as Microsoft® HealthVault™ and Google™ Health can be used by the patient 100 and clinician 102 to exchange information.

The diabetes manager 104 can receive glucose readings from one or more sources (e.g., from the CGM 200). The CGM 200 continuously measures the glucose level in the abdominal interstitial fluid of the patient 100. The CGM 200 periodically communicates the readings to the diabetes manager 104. The diabetes manager 104 and the CGM 200 communicate wirelessly using a proprietary Gazell wireless protocol developed by Nordic Semiconductor, Inc.

Additionally, the diabetes manager 104 includes a blood glucose meter (BGM) and a port that communicates with the BGM (not shown). The port can receive a blood glucose measurement strip 306. The patient 100 deposits a sample of blood on the blood glucose measurement strip 306. The BGM analyzes the sample and measures the blood glucose level in the sample. The blood glucose level measured from the sample can be used to calibrate the interstitial glucose level from the CGM 200 in order to estimate a BG value from a CGM value. BG values can be used to determine the amount of insulin to be administered to the patient 100.

The diabetes manager 104 communicates with the insulin pump 202 or 204. The insulin pump 202 or 204 can be configured to receive instructions from the diabetes manager 104 to deliver a user-determined amount of insulin to the patient 100. Additionally, the diabetes manager 104 can receive other information including meal and/or exercise schedules of the patient 100 and use these to determine the amount of insulin to administer using insulin pump 202 or 204 based on the additional information.

The insulin pump 202 or 204 can also communicate data to the diabetes manager 104. The data can include amounts of insulin delivered to the patient 100, corresponding times of delivery, and pump status. The diabetes manager 104 and the insulin pump 202 or 204 can communicate using a wireless communication protocol such as Bluetooth. Other wireless or wireline communication protocols can also be used.

In addition, the diabetes manager 104 can communicate with the other healthcare devices 304. For example, the other healthcare devices 304 can include a blood pressure meter, a weight scale, a pedometer, a fingertip pulse oximeter, a thermometer, etc. The other healthcare devices 304 obtain and communicate personal health information of the patient 100 to the diabetes manager 104 through wireless, USB, or other interfaces. The other healthcare devices 304 may use communication protocols compliant with ISO/IEEE 11073 extended using guidelines from Continual® Health Alliance. The diabetes manager 104 can communicate with the other healthcare devices 304 using interfaces including Bluetooth, USB, etc. Further, the devices of the diabetes management system 300 can communicate with each other via the diabetes manager 104.

The diabetes manager 104 can communicate with the PC 106 using Bluetooth, USB, or other interfaces. A diabetes management software running on the PC 106 includes an analyzer-configurator that stores configuration information of the devices of the diabetes management system 300. The configurator has a database to store configuration information of the diabetes manager 104 and the other devices. The configurator can communicate with users through standard web or computer screens in non-web applications. The configurator transmits user-approved configurations to the devices of the diabetes management system 300. The analyzer retrieves data from the diabetes manager 104, stores the data in a database, and outputs analysis results through standard web pages or computer screens in non-web based applications.

The diabetes manager 104 can communicate with the mobile device 302 using Bluetooth. The mobile device 302 may include a cellular phone, a pager, or a personal digital assistant (PDA). The diabetes manager 104 can send messages to an external network through the mobile device 302. The mobile device 302 can transmit messages to the external network upon receiving requests from the diabetes manager 104.

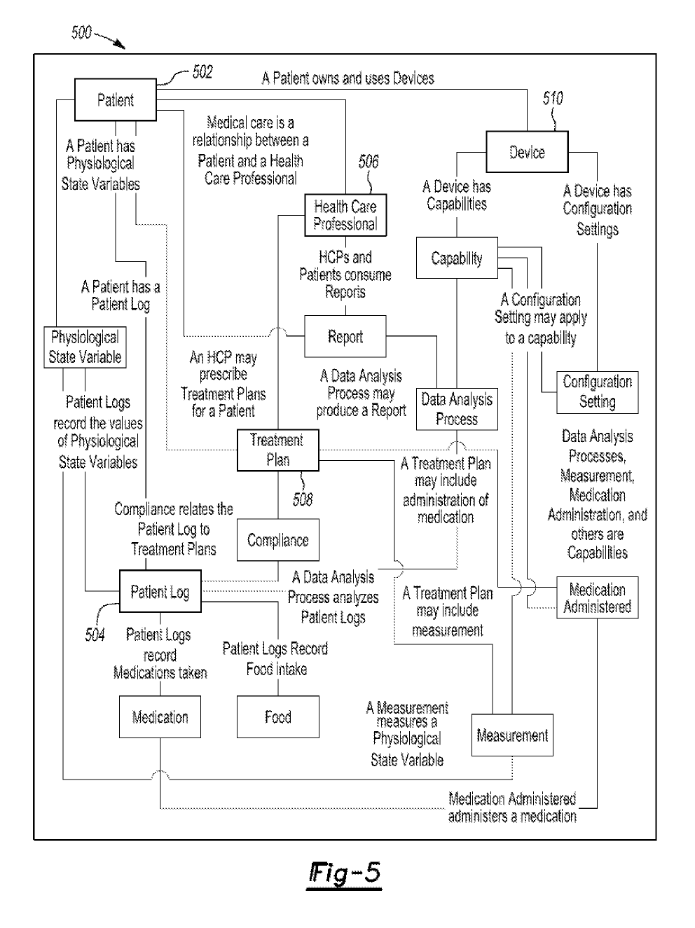

Referring now to FIG. 4, the diabetes manager 104 comprises a blood glucose measuring (BGM) module 400, a communication module 402, a user interface module 404, user interfaces 406, a processing module 408, memory 410, and a power module 412. The BGM module 400 includes a blood glucose measuring engine that analyzes samples provided by the patient 100 on the blood glucose measurement strip 306 and that measures the amount of blood glucose in the samples. The communication module 402 includes multiple radios that communicate with different devices of the diabetes management system 300, as well as communication processors to implement the communication protocols. The user interface module 404 interfaces the diabetes manager 104 to various user interfaces 406 that the patient 100 can use to interact with the diabetes manager 104. For example, the user interfaces 406 can include keys, switches, a display, a speaker, a microphone, a secure digital (SD) card port, a USB port, etc. (not shown).

The processing module 408 processes data received from the BGM module 400, the communication module 402, and the user interface module 404. The processing module 408 uses memory 410 for processing and storing data. The memory 410 can include volatile and nonvolatile memory. The processing module 408 outputs data to and receives data from the user interfaces 406 via the user interface module 404. The processing module 408 outputs data to and receives data from the devices of the diabetes management system 300 via the communication module 402. The power module 412 supplies power to the components of the diabetes manager 104. The power module 412 includes a rechargeable battery. The battery can be recharged using an adapter that plugs into a wall outlet. The battery can also be charged via the USB port of the diabetes manager 104.

FIG. 5 depicts a domain model 500 for the diabetes care information management domain. A domain model is a conceptual model of a system's domain of application (in this case diabetes care) which describes the entities in the domain and their relationships. In the area of diabetes care, the domain model 500 is built around a patient 502 who is receiving medical care for diabetes. Each patient 502 has an associated patient log 504 for recording actions taken by or in relation to the patient. Exemplary actions may include recording medication intake, food intake, or values of a patient's physiological state variable such as a blood glucose measure. Medical care is provided to the patient under the direction of one more health care professionals 506. More specifically, a health care professional 506 may prescribe a treatment plan 508 comprised of a series of actions for administering medical treatment to the patient. Administering medical care typically involves one or more medical devices 510 having different capabilities and configuration settings. It is readily understood that the domain model may include other entities and relationships related to diabetes care.

A domain model has two primary uses. First, a shared domain model insures that stakeholders and developers are thinking about the problem at hand in the same way and using the same terminology. Second, a domain model is used for identifying variability for architecture and design. Since a domain model is expressed in terms of the application area, variability points identified in the model are more likely to have business value. When technical architects and designers are aware of this predicted variability, they are better able to produce designs that will evolve in ways useful to the business. In this disclosure, the diabetes care information management domain model 50 is used to identify business-relevant extension points for a computer-implemented diabetes care system.

First, a discussion is provided to clarify what is meant by extensibility and extension points. There are several meaningful ways to define extensibility. For the purpose of this disclosure, four types of extensibility are specifically defined for software and firmware: design extensible, compile-time extensible, run-time extensible and dynamically extensible.

A particular architecture or design is design extensible relative to a domain concept X if a new variant or member of X can be added to the system without requiring any re-design, where “re-design” is any change that requires a revision any of the system's architecture or high-level design documents. Design extensibility is the simplest version of extensibility. Note that under this definition, requiring a complete system re-build or even substantial code changes is permitted, as is added detailed design documentation for new elements.

A particular architecture or design is compile-time extensible relative to a domain concept X if it is design extensible relative to X and if adding a new variant or member for X can be done by a) adding code for the new member/variant and b) adding code in one place to include the new member/variant and c) recompiling all or part of the system.

A particular architecture or design is run-time extensible relative to a domain concept X if it is design extensible relative to X and if adding a new variant or member for X can be done by building a new module, locating it in a design-specified place, and modifying a manifest or other (non-code) configuration information. Run-time extensibility may require that the system be re-started before the new concept is visible in the system. Note that run-time extensibility does not require compile-time extensibility; these two extensibility methods are mutually exclusive.

For completeness, a definition for dynamic extensibility is included, which is characteristic of service-oriented architectures with service discovery. A particular architecture or design is dynamically extensible relative to a domain concept X if it is design extensible relative to X and if adding a new variant or member for X can be done dynamically while the system remains running.

Extensibility typically requires an explicit representation of the identified concept. In an exemplary embodiment, the diabetes management system 300 is implemented using an object-oriented programming paradigm. In an object-oriented system, the explicit representation will be a class. Such abstractions should be isolated to a component intended for abstractions (i.e., not mixed with implementations). In this disclosure, the class diagrams are defined in accordance with the Unified Modeling Language (UML).

While this disclosure makes reference primarily to an object-oriented programming paradigm, it is readily understood that other types of implementations fall within the scope of this disclosure. For example, the diabetes management system 300 may be implemented using functional designs. In functional designs, the explicit representation will typically be as a structure (record) type, with functions that work on that type isolated to a single module or component. If code that knows about a type is scattered across multiple modules, this is a sign that the given structure is not extensible. In a functional implementation, the header files declaring the structure type and externally-visible functions operating on that type should be isolated. Other types of implementations of the system are also contemplated.

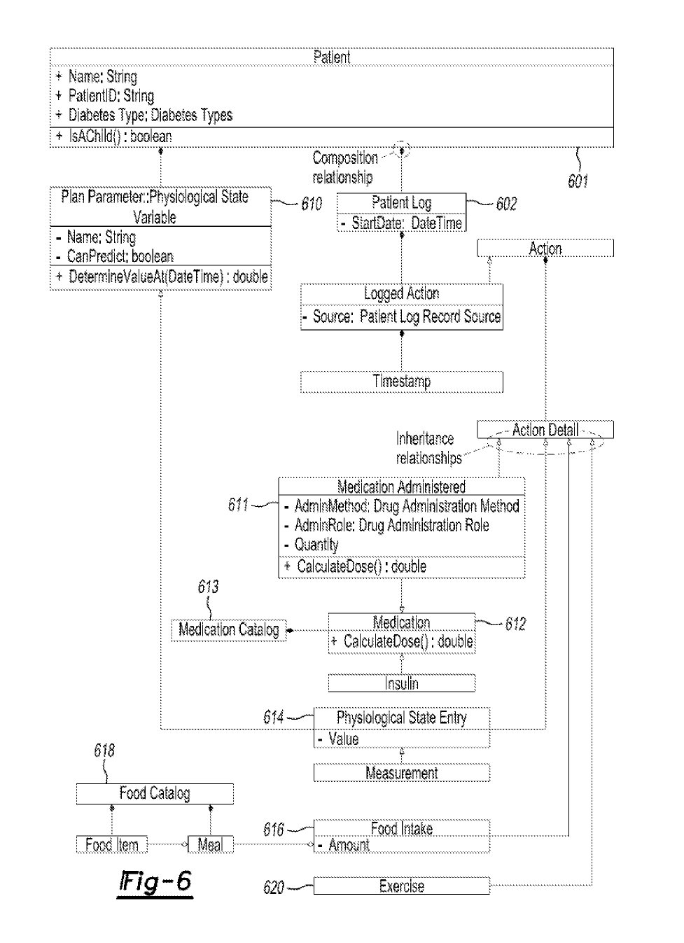

FIG. 6 is a class diagram for a portion of the diabetes care domain model that relates to a patient's log. Each patient has associated patient logs for recording a series of actions taken by or in relation to the patient as noted above. Patient logs are an identified extension point. Accordingly, the patient log class 602 has a composition relationship with the patient class 601; the patient class 601 does not know anything about the contents of this composition except that they embody the patient log concept. Attributes of the patient log class 602 include (but are not limited to) the date of the first entry in the log. Entries in the log are represented by the objects of the “logged action” class, which include (but are not limited to) an identifier for the origin of an action (e.g., a manual entry or a device) and a timestamp when the action occurred. Logged actions are an identified extension point. The patient log class 602 is composed of logged actions; the patient log 602 does not know anything about the contents of this composition except that they embody the logged action concept. In the exemplary embodiment, the logged actions are one of four different types: medication administration 611, physiological state entry 614, food intake 616 or exercise 620. Each of these action types is a subclass to a superclass that is associated with the logged action class as shown in the figure. Other types of actions are also contemplated by this disclosure.

The medication administered class 611 records the administration of medicine to the patient. The medication class 612 is associated with the medication administration class (i.e., each medication administration object has a relationship to a medication object for the medication that was taken by the patient). Supporting new types of medication is an identified extension point. Accordingly, the medication catalog class 613 has a composition relationship with the medication class 612; the medication catalog class 613 does not know anything about the contents of this composition except that they embody the medication concept. Supporting new ways to calculate dosages for a given medication is another identified extension point. Thus, the medication class further includes a method that calculates a dosage of the medication to be administered to the patient (e.g., a bolus insulin dosage calculation).

The physiological state variable entry class 614 refers to an input of a value of a physiological state variable such as blood glucose or a patient's wake/sleep state. Supporting new types of physiological state variables in the system is another identified extension point. Accordingly, the patient class 601 has a composition relationship with the physiological state variable class 610; the patient class 601 does not know anything about the contents of this composition except that they embody the physiological state variable concept. For example, the glycemic index is a measure of how much your blood glucose level swings up and down. Supporting such physiological variables and methods for calculating the same are anticipated in future developments. Extensibility for these types of new variables is supported by defining a new subclass or instance of physiological state variable class 610. Determining the value of a physiological state variable from date in the patient log 602 is a defined extension point. Thus the physiological state variable class 610 further has a method for determining its value at a particular date and time.

The food intake class 616 represents food the patient has consumed. Food intake is associated with a meal object, which aggregates food items. Supporting new types of food in the system is another identified extension point. Accordingly, the food catalog class 618 has a composition relationship with food items; the food catalog class 618 does not know anything about the contents of this composition except that they embody the food item concept. New calculation logic associated with food items is also an identified extension point. For example, the rate at which foods introduce glucose into the bloodstream depends in part on the fat content of the food; new calculation logic for food items could include (but is not limited to) the duration over which the carbohydrate content should be distributed when making predictions.

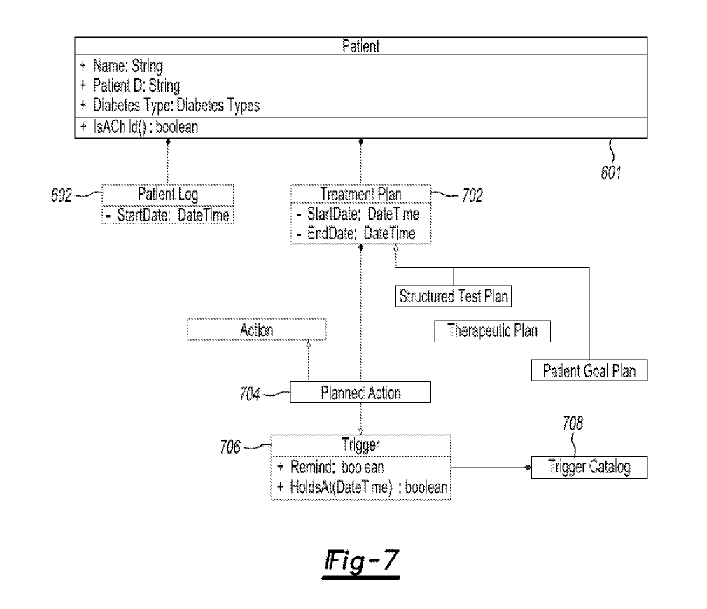

FIG. 7 depicts a portion of the diabetes care domain model that relates to a treatment plans. A treatment plan is a general concept that covers a wide variety of planned actions on the part of the patient. In the context of diabetes care, a treatment plan is understood to be a series of planned action related to the medical treatment of the patient. Treatment plans may be a structured testing plan like a 3-day profile, and therapeutic plan like a pump's basal profile, a user goal plan like a target weight, or other types of plans not yet envisioned. Supporting new types of treatment plan is an identified extension point. Accordingly, the patient class 601 has a composition relationship with treatment plans 702; the patient class 601 does not know anything about the contents of this composition except that they embody the treatment plan concept. Attributes of the treatment plan class include (but are not limited to) a starting date, an ending date and a series of planned actions which comprise the treatment plan.

An important aspect of a treatment plan is the capability to define the conditions that trigger one of the planned actions. A reminder to take a given medication based on the time of day or a reminder to perform a test based on a time interval since a given meal are examples of triggers. Supporting new types of triggers is an identified extension point. Accordingly, the trigger catalog class 708 has a composition relationship with the treatment plan trigger class 706; the trigger catalog class 708 does not know anything about the contents of this composition except that they embody the treatment plan concept. Actions in a treatment plan are instances of the planned action class 704; each such action is associated with a trigger from the trigger catalog. In an exemplary embodiment, the diabetes management system 300 provides run-time extensibility for ne types of triggers in the manger set forth below. New ways to compute trigger conditions are an identified extension point. Thus the trigger class 706 further has a method for determining whether the trigger holds at a particular date and time.

A structured test plan is a particular type of treatment plan defined in the diabetes care domain. For example, a structured test plan may be a series of planned actions for obtaining measurement data from a glucose meter. In addition to a series of planned actions, the structured test plan includes attributes such as an entry criterion, an adherence criterion and an exit criterion. Entry criteria establish the conditions that need to be met prior to obtaining a physiological variable measure from the patient; this corresponds to the trigger class 706. Adherence criteria are used to assess qualitatively whether a planned action was performed. Exit criterian establish the conditions needed to be met prior to exiting the structured test plan.

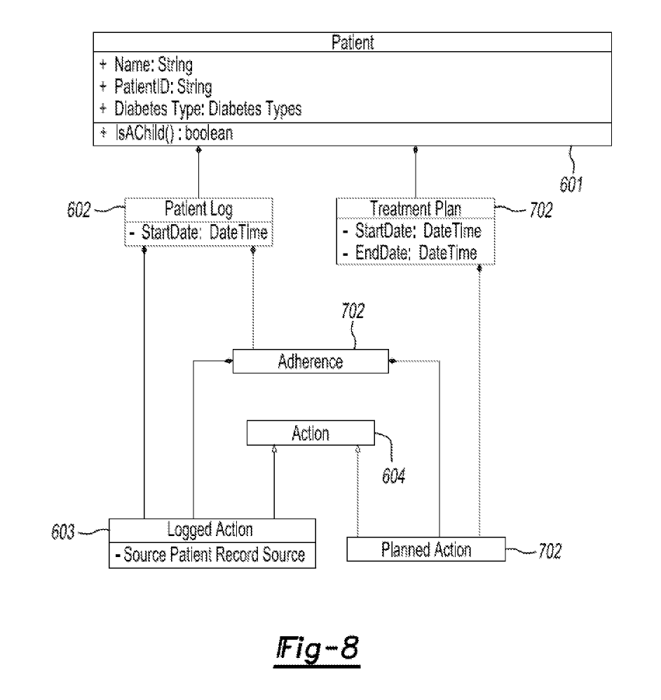

Adherence information for the patient may be managed using an adherence class 702 as shown in FIG. 8. More specifically, the adherence class 702 creates a relationship between planned actions for the patient defined by a treatment plan and the actions taken by the patient as recorded in the patient log. In an exemplary embodiment, the adherence class 702 may define an indicator having values, such as compliant or non-compliant, as well as adherence criteria or methods for deriving a value for the indicator. In this example, the indicator may be assigned a ‘compliant’ value when an action planned for a particular time is performed within a prescribed range (as defined by the adherence criteria). Supporting new types of adherence is an identified extension point. Accordingly, the patient log class 602 has a composition relationship with the adherence class 702; the patient log class 602 does not know anything about the contents of this composition except that they embody the adherence concept.

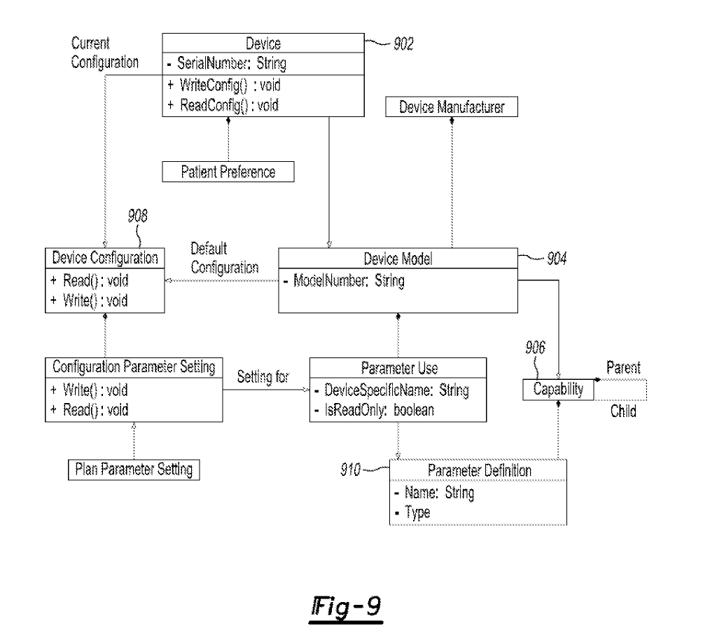

FIG. 9 depicts a portion of the diabetes care domain model that relates to medical devices. Managing diabetes typically involves one or more medical devices. Over time, devices having new capabilities and/or configuration settings will emerge. Supporting new types of device capabilities and configuration parameters are identified extension points. Accordingly, the capability class 906 forms a hierarchical (parent-child) structure using composition; the parent capability does not know anything about the contents of this composition except that they embody the capability. A particular device model implements some set of capabilities, as shown by the many-to-many relationship between the device model class 904 and the capability class 906. Similarly, the capability class 906 has a composition relationship with the parameter definition class 910; the capability does not know anything about the contents of this composition except that they embody the parameter definition concept. The relationships from device 902 to device model 904 and from device model 902 to device configuration 908 promote extensibility by insuring that configurations for individual devices are expressed in terms of the extensible concepts capability 906 and parameter definition 910.

Extensibility requires that an explicit representation of the extensible concept can be modified in some way that does not impact the design. In object-oriented systems, this is typically done with inheritance or with a container class referencing an interface. In functional systems, this can be done with a union structure or function pointers, or at worst with untyped pointers. Other techniques for changing the explicit representation are also contemplated by this disclosure.

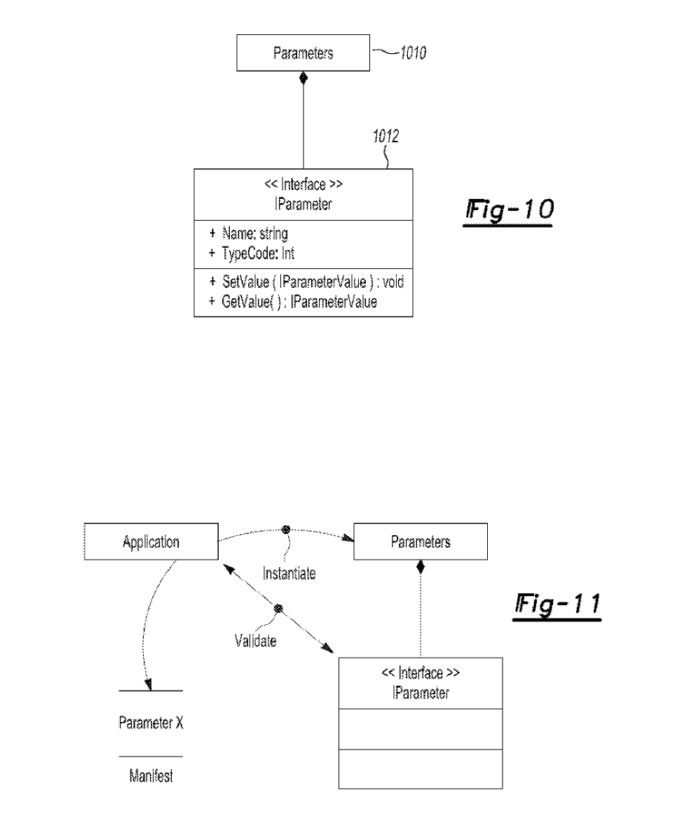

FIG. 10 illustrates a partial example of a detailed design using a container class 1010 referencing an interface 1012. For illustration purposes, supporting new parameters, such as device parameters, is the identified extensibility point. The design includes a ‘Parameters’ container (or containment) class having a composition relationship with an ‘1Parameter’ interface. The ‘Parameter’ container class contains all of the system defined device parameters. An interface is an abstract class that specifies the elements, such as attributes and methods, common to all parameters of a device. In this simplified example of the ‘IParameter’ interface, each of the device parameters must have a name attribute and a typecode attribute. In addition, each of the device parameters must provide a method for setting the parameter value and a method for retrieving the parameter value. Additional attributes and/or method may be needed for an actual system design. Whether the members of this containment relationship are established at compile-time or run-time determines whether this design implements compile-time extensibility or run-time extensibility. In any case, it is understood that this principle can be extended to other data types.

For run-time extensibility, the system needs to be able to access software modules that were not implemented when the system was initially built. In the exemplary embodiment, the system has been implemented on devices which utilize the Microsoft Windows CE operating system. Thus, dynamic link loading is used by the system to access new software modules or libraries. In other computing environments, an interpreter or some other form of remote call may be used to access new software modules at run-time.

With reference to FIG. 11, an application may also want to make use of a new data types, such as a new device parameter. In this case, run-time extensibility requires that the application have some external source of knowledge about the new device parameter. In an exemplary embodiment, the new device parameter, such as Parameter X, is defined in a manifest. The manifest may be represented in a variety of ways, including binary file, text file, database, registry entry, etc. XML is a preferred format for the manifest representation.

At system start up, the application will instantiate one or more objects in the Parameter container class by referencing the ‘IParameter’ interface. For example, the application may make use of Parameter X. This new device parameter as defined in the manifest will be validated against the ‘IParameter’ interface. The new device parameter is instantiated as an object in the Parameter container class only if the new device parameter meets the criteria set forth by the interface. In this way, extensibility of new data types may be supported in the system. Instantiating new objects in this manner is one exemplary means of instantiating objects using a single coded list, such as a manifest, in a computer memory. Other means for instantiating objects using a single coded list also fall within the scope of this disclosure.

In an exemplary embodiment, a handheld medical device that supports extensibility for diabetes care would be set up as follows. First, an instance of the patient class with collections (compositions) for patient logs, physiological state variables, and treatment plans is created. Similarly, an instance of the medication catalog, trigger catalog and root capability will be created with collections for medications, treatment plan triggers, and child capabilities, respectively. Base classes for logged actions and adherence would also exist in the default configuration, but may not have instances in the initial (default) setup. Constructs for and the relationships amongst these classes are set forth above. In one implementation, extensibility requirements are enforced through inheritance. For example, all members of the patient object's physiological state variables collection would be derived from a common physiological state variable base class. In addition to those discussed above, it is readily understood that the device may be configured with other classes needed to support the functionality of the device.

The medical device is preferably configured with a meter that measures concentration of glucose in blood or some other type of measurement component (which may be no more than a way for users to manually enter a measurement value). The medical device further includes at least one application that utilizes the class structure. More specifically, the application instantiates an object from at least one of the patient class, the patient log class, the treatment plan class and the adherence class and performs a function using the instantiated objects. In the exemplary embodiment, the application is comprised of computer executable instructions executed by a computer processor in the device. While the extensibility configuration has been described in the context of a single medical device, it is understood that these principles apply to a diabetes management system distributed across multiple devices and/or having multiple software applications.

In another implementation, extensibility requirements are enforced by implementing select classes as container classes referencing an interface as described above. For example, the patient class would have containers for treatment plans and physiological state variables. In this example, a treatment plan interface a physiological state variable interface are also defined in the memory of the device. The treatment plan interface is an abstraction class that specifies the methods and properties that any member of the treatment plans collection must support. As shown in FIG. 7, this would include (but is not limited to) supporting properties for start date and end date. Similarly, the physiological state variable interface that specifies the methods and properties that any member of the physiological state variables collection must support. As shown in FIG. 6, this would include (but is not limited to) support properties for the variable's name and whether the variable's value can be predicted in the future, plus a method for calculating the value based on data in the patient logs. In this implementation, the application instantiates an object from some concrete class that supports the interface, for example by using a manifest as discussed above. Other classes in the domain model may also be implemented as container classes referencing an interface.

For purposes of clarity, the same reference numbers will be used in the drawings to identify similar elements. As used herein, the phrase at least one of A, B, and C should be construed to mean a logical (A or B or C), using a non-exclusive logical or. It should be understood that steps within a method may be executed in different order without altering the principles of the present disclosure.

As used herein, the term module may refer to, be part of, or include an Application Specific Integrated Circuit (ASIC); an electronic circuit; a combinational logic circuit; a field programmable gate array (FPGA); a processor (shared, dedicated, or group) that executes code; other suitable components that provide the described functionality; or a combination of some or all of the above, such as in a system-on-chip. The term module may include memory (shared, dedicated, or group) that stores code executed by the processor.

The term code, as used above, may include software, firmware, and/or microcode, and may refer to programs, routines, functions, classes, and/or objects. The term shared, as used above, means that some or all code from multiple modules may be executed using a single (shared) processor. In addition, some or all code from multiple modules may be stored by a single (shared) memory. The term group, as used above, means that some or all code from a single module may be executed using a group of processors. In addition, some or all code from a single module may be stored using a group of memories.

The apparatuses and methods described herein may be implemented by one or more computer programs executed by one or more processors. The computer programs include processor-executable instructions that are stored on a non-transitory tangible computer readable medium. The computer programs may also include stored data. Non-limiting examples of the non-transitory tangible computer readable medium are nonvolatile memory, magnetic storage, and optical storage.

What is claimed is:

1. A medical device that supports extensibility for diabetes care, comprising:
a patient class implemented in the computer memory of the device, where the patient class has properties and methods associated with a person receiving medical treatment for diabetes;
one or more patient log classes implemented in the computer memory of the device, where the patient log classes have properties and methods associated with a persistent log of actions taken by the patient and are in a composition relationship with the patient class; and
an application that instantiates a patient object from the patient class and fills a patient log composition of the patient class with patient log objects instantiated from the patient log class or subclasses thereof by means of a single coded list in computer memory, where the application uses the instantiated patient object and instantiated patient log objects to perform functions, and the application is computer executable instructions executed by a computer processor in the device.

2. The medical device of claim 1 further comprises a logged action class implemented in the computer memory of the device, where the logged action class has properties and methods associated with entries in a patient log and is in a composition relationship with the patient log class; where the application further instantiates logged action objects of the patient log composition by means of a single coded list in computer memory where the logged action objects instantiated are instances of the logged action class or subclasses thereof.

3. The medical device of claim 2 wherein the logged action class includes a timestamp and supports logging medication administration, values of physiological state variables, food intake, and exercise, where the application further uses the instantiated logged action objects to modify entries in patient logs.

4. The medical device of claim 2 further comprises a physiological state variable class implemented in the computer memory of the device, where the physiological state variable class has properties and methods associated with measurable or recordable properties of a patient's physiology, and is in a composition relationship with the patient class; the application further instantiates objects of a physiological state variable composition by means of a single coded list in computer memory where the objects instantiated are instances of the physiological state variable class or subclasses thereof; and the application supports the creation, entry or modification of logged actions corresponding to membership of the physiological state variables composition of the patient class.

5. The medical device of claim 4 further comprises
a medication catalog class implemented in the computer memory of the device; and
a medication class implemented in the computer memory of the device, where medication class has properties and methods associated with drugs or medicines, and is in a composition relationship with a medication catalog class; the application further instantiates objects of a medication composition of the medication catalog class by means of a single coded list in computer memory where the objects instantiated are instances of the medication class or subclasses thereof; and the application supports the creation, entry or modification of logged actions corresponding to membership of the medications composition of the medication catalog class.

6. The medical device of claim 5 wherein the medication class has a method for calculating the dosage of medication from data in the patient logs and knowledge of a specific medication, and said method for calculating dosage is used to make recommendations to the patient on dosage.

7. The medical device of claim 6 further comprises:
one or more treatment plan classes implemented in the computer memory of the device, where the treatment plan classes have properties and methods associated with a series of planned actions related to therapy to be undertaken by the patient, and are in a composition relationship with the patient class; and
an application that instantiates an object from the patient class and fills a treatment plan composition of the patient class with treatment plan objects instantiated from the treatment plan class or subclasses thereof by means of a single coded list in computer memory, where the application uses the instantiated patient object and instantiated treatment plan objects to perform functions, and the application is computer executable instructions executed by a computer processor in the device.

8. The medical device of claim 7 further comprises
a trigger catalog class implemented in the computer memory of the device; and
a trigger class implemented in the computer memory of the device, the trigger class has properties and methods associated with triggering a planned actions in a treatment plan, and is in a composition relationship with the trigger catalog class;
the application further instantiates objects of a trigger composition of the trigger catalog class by means of a single coded list in computer memory where the objects instantiated are instances of a trigger class or subclasses thereof, and the application supports the creation, entry, modification and use of planned actions that are associated with the trigger composition of the trigger catalog class.

9. The medical device of claim 8 further comprises
an adherence class implemented in the computer memory of the device, the adherence class has properties and methods associated with how well actions taken by a patient match the planned actions of a given treatment plan, and is in a composition relationship with the patient log class;
the application further instantiates objects of a adherence composition of the patient log class by means of a single coded list in computer memory where the objects instantiated are instances of an adherence class or subclasses thereof, and the application supports the creation, entry, modification and use of planned actions and logged actions that are associated with the objects of the adherence composition of the patient log class.

10. The medical device of claim 1 further comprises
an interface definition in the computer memory of the device which identifies names and types of the properties and methods to be supported by objects of the patient log composition; and
an external manifest that identifies libraries, classes, and instances to be used to fill the patient log composition, where the application uses the manifest to create patient log objects to populate the patient log composition.

11. The medical device of claim 1 further comprises a meter that measures blood glucose.

12. A medical device that supports extensibility for diabetes care, comprising:
a patient class implemented in the computer memory of the device, where the patient class has properties and methods associated with a person receiving medical treatment for diabetes;
one or more treatment plan classes implemented in the computer memory of the device, where the treatment plan classes have properties and methods associated with a series of planned actions related to therapy to be undertaken by the patient, and are in a composition relationship with the patient class; and
an application that instantiates an object from the patient class and fills a treatment plan composition of the patient class with treatment plan objects instantiated from the treatment plan class or subclasses thereof by means of a single coded list in computer memory, where the application uses the instantiated patient object and instantiated treatment plan objects to perform functions, and the application is computer executable instructions executed by a computer processor in the device.

13. The medical device of 12 further comprises
a trigger catalog class implemented in the computer memory of the device; and
a trigger class implemented in the computer memory of the device, the trigger class has properties and methods associated with triggering a planned actions in a treatment plan, and is in a composition relationship with the trigger catalog class;
the application further instantiates objects of a trigger composition of the trigger catalog class by means of a single coded list in computer memory where the objects instantiated are instances of a trigger class or subclasses thereof, and the application supports the creation, entry, modification and use of planned actions that are associated with the trigger composition of the trigger catalog class.

14. The medical device of claim 13 further comprises:
an adherence class implemented in the computer memory of the device, the adherence class has properties and methods associated with how well actions taken by a patient match the planned actions of a given treatment plan, and is in a composition relationship with the patient log class;
the application further instantiates objects of a adherence composition of the patient log class by means of a single coded list in computer memory where the objects instantiated are instances of an adherence class or subclasses thereof, and the application supports the creation, entry, modification and use of planned actions and logged actions that are associated with the objects of the adherence composition of the patient log class.

15. The medical device of claim 12 further comprises
one or more patient log classes implemented in the computer memory of the device, where the patient log classes have properties and methods associated with a persistent log of actions taken by the patient and are in a composition relationship with the patient class; and
the application further instantiates a patient object from the patient class and fills a patient log composition of the patient class with patient log objects instantiated from the patient log class or subclasses thereof by means of a single coded list in computer memory, where the application uses the instantiated patient object and instantiated patient log objects to perform functions.

16. The medical device of claim 15 further comprises a logged action class implemented in the computer memory of the device, where the logged action class has properties and methods associated with entries in a patient log and is in a composition relationship with the patient log class; where the application further instantiates logged action objects of the patient log composition by means of a single coded list in computer memory where the logged action objects instantiated are instances of the logged action class or subclasses thereof.

17. The medical device of claim 16 wherein the logged action class includes a timestamp and supports logging medication administration, values of physiological state variables, food intake, and exercise, where the application further uses the instantiated logged action objects to modify entries in patient logs.

18. The medical device of claim 16 further comprises a physiological state variable class implemented in the computer memory of the device, where the physiological state variable class has properties and methods associated with measurable or recordable properties of a patient's physiology, and is in a composition relationship with the patient class; the application further instantiates objects of a physiological state variable composition by means of a single coded list in computer memory where the objects instantiated are instances of the physiological state variable class or subclasses thereof; and the application supports the creation, entry or modification of logged actions corresponding to membership of the physiological state variables composition of the patient class.

19. The medical device of claim 18 further comprises
a medication catalog class implemented in the computer memory of the device; and
a medication class implemented in the computer memory of the device, where medication class has properties and methods associated with drugs or medicines, and is in a composition relationship with a medication catalog class; the application further instantiates objects of a medication composition of the medication catalog class by means of a single coded list in computer memory where the objects instantiated are instances of the medication class or subclasses thereof; and the application supports the creation, entry or modification of logged actions corresponding to membership of the medications composition of the medication catalog class.

20. The medical device of claim 19 wherein the medication class has a method for calculating the dosage of medication from data in the patient logs and knowledge of a specific medication, and said method for calculating dosage is used to make recommendations to the patient on dosage.

21. The medical device of claim 12 further comprises
an interface definition in the computer memory of the device which identifies names and types of the properties and methods to be supported by objects of the patient log composition; and
an external manifest that identifies libraries, classes, and instances to be used to fill the patient log composition, where the application uses the manifest to create patient log objects to populate the patient log composition.

22. The medical device of claim 12 further comprises a meter that measures blood glucose.

23. A medical device that supports extensibility, comprising:
a device model class implemented in the computer memory of the device, the device model class has properties and methods associated with a particular model of medical device;
one or more capability classes implemented in the computer memory of the device, the capability classes have properties and methods associated with a particular capability of a medical device, and are in a composition relationship with a root capability class.
an application that instantiates an object from the device model class and fills a capability composition of the device class with objects instantiated by means of a single coded list in computer memory where the objects instantiated are instances of the capability class or subclasses thereof;
the application uses the instantiated device model object and the instantiated capability objects to perform functions, wherein the application is computer executable instruction executed by a computer processor in the device.

24. The device of claim 23 further comprises:
a parameter definition class implemented in the computer memory of the device, the parameter definition class has properties and methods associated with device configuration parameters, and is in a composition relationship with the capability class;
the application further instantiates objects of the parameter definition composition of the capability class by means of a single coded list in computer memory where the objects instantiated are instances of a parameter definition class or subclasses of that class, and the application uses said instantiated parameter definition composition to support editing configuration parameters based on the objects of the parameter definition compositions of the capability classes.
